(19) United States
(12) Patent Application Publication
(10) Pub. No.: US 2006/0216327 A1
(43) Pub. Date: Sep. 28, 2006

(54) Multilayer coating for releasing biologically-active agents and method of making

(75) Inventors: Todd A. Madsen, Bozeman, MT (US); Jamal S. Yanaki, Salt Lake City, UT (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Bacterin, Inc., Belgrade, MT (US)

(21) Appl. No.: 11/091,354

(22) Filed: Mar. 28, 2005

(51) Int. Cl. A61K 9/26

(52) U.S. Cl. 424/426; 424/470

(57) ABSTRACT

A composition for use with healthcare products such as medical device includes one or more biologically-active agents for release into bodily fluids. The composition can be applied to a substrate to form an erodible layer and a polymer layer. Biologically-active agent(s) are releasable from the erodible layer and the polymer layer when exposed to aqueous fluids.

### BACKGROUND OF THE INVENTION

The present invention relates generally to compositions for site-specific or localized delivery of biologically-active agents. More specifically, the present invention relates to a multi-layer coating for use with medical devices or other healthcare products to release biologically-active agents in an aqueous fluid.

The development of compositions for localized or site-specific delivery of biologically-active agents is a rapidly developing area of medicine. For example, medical devices such as coronary stents have increasingly employed drug-eluting coatings to deliver drugs directly to areas surrounding the implanted medical device to help minimize some of the harmful effects (such as host inflammation) associated with the implanted medical device.

The ability to deliver drugs in a site-specific or local manner has the potential to provide various benefits. For example, it may ensure that therapeutic amounts of biologically-active agents reach desired treatment sites and may avoid side effects that can occur through systematic delivery.

### BRIEF SUMMARY OF THE INVENTION

The present invention includes compositions and articles for releasing one or more biologically-active agents into an aqueous fluid, as well as methods for making the compositions and articles. In one embodiment, the present invention is directed to a composition that includes a release material that disintegrates in an aqueous fluid, a first biologically-active agent incorporated within the release material, a polymer matrix, and a second-biologically active agent incorporated within the polymer matrix. The first and second biologically-active agents are releasable from the composition upon exposure to an aqueous fluid.

In another embodiment, the present invention is directed to an article including a substrate, an erodible layer, and a polymer layer. The erodible layer is located on a surface of the substrate and includes a release material and a first biologically-active agent that is releasable from the erodible layer when exposed to an aqueous fluid. The polymer layer includes a polymer and a second biologically-active agent releasable from the erodible layer when exposed to the aqueous fluid.

In another embodiment, the present invention is directed to a method for coating a substrate. The method includes providing functional groups on a surface of the substrate. A base layer, which includes a release material and a first biologically-active agent, is deposited on the functionalized surface. The base layer is coated with a polymer layer that includes a second biologically-active agent that is diffusible from the polymer layer when exposed to an aqueous fluid.

### BRIEF DESCRIPTION OF THE DRAWINGS

FIG. 1 is a diagram showing a cross-sectional view of a coating of the present invention releasing biologically-active agents.

FIG. 2A is a scanning electron micrograph of a stent prepared as described in Example 3 below and including a calcium phosphate base layer applied to the stent.

FIG. 2B is a scanning electron micrograph of a stent prepared as described in Example 4 below and including a poly(vinyl alcohol) polymer layer applied to the calcium phosphate base layer of FIG. 2A.

FIG. 3 is a graph showing release of bupivacaine from the stent of FIG. 2B.

### DETAILED DESCRIPTION

The present invention includes compositions that release one or more biologically-active agents when exposed to an aqueous fluid. The compositions include a release material, one or more biologically-active agents, and a polymer. The compositions may be formed into coatings capable of affecting biological processes by releasing biologically-active agent(s). Coatings of the present invention include an erodible layer with one or more biologically-active agents incorporated therein and a polymer layer positioned over the erodible layer and also having one or more biologically-active agents incorporated therein.

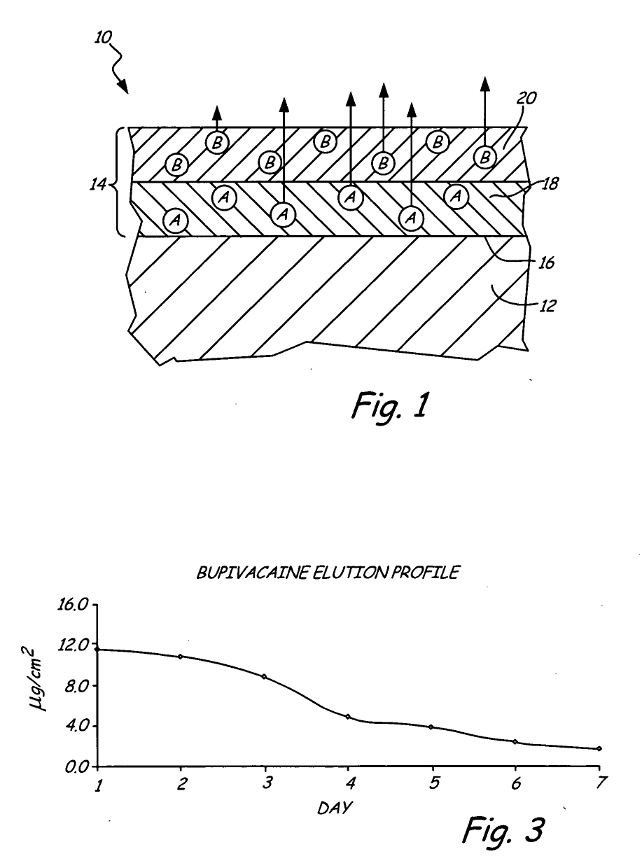

FIG. 1 shows a diagram illustrating article 10, which includes substrate 12 and coating 14. Surface 16 of substrate 12 is coated with coating 14, which includes base layer 18 and polymer layer 20. As shown in FIG. 1, base layer 18 and polymer layer 20 are in contact with each other, with base layer 18 covering surface 16 and polymer layer 20 covering base layer 18. Base layer 18 and polymer layer 20 can each be of any thickness and can each include any number of constituent layers.

Coating 14 can include layers of material in addition to base layer 18 and polymer layer 20. In some embodiments, one or more layers of material are located between base layer 18 and polymer layer 20 and/or between surface 16 and base layer 18. In some embodiments, one or more layers of material overlie polymer layer 20.

Base layer 18 and polymer layer 20 each include at least one biologically-active agent. As shown in FIG. 1, base layer 18 includes biologically-active agent A and polymer layer 20 includes biologically-active agent B. Upon exposure of article 10 to an aqueous fluid, biologically-active agent A is released from base layer 18 and biologically-active agent B is released from polymer layer 20. As used herein, an “aqueous fluid” is defined to include any fluid naturally present in a mammalian body (“bodily fluid”), any fluid containing water, and any fluid in which water is a solvent. In some embodiments, base layer 18 and polymer layer 20 exhibit different release kinetics for biologically-active agents A and B.

As used herein, a “biologically-active agent” means a substance that, when placed in contact with a living organism, affects a biological process in a manner that produces a detectable result (other than instigating a foreign body response). Examples of biologically-active agents for use in the present invention include analgesics, antimicrobials (e.g., antibiotics, antifungals, or antivirals), anti-inflammatories, targeting agents, cytokines, immunotoxins, antihistamines, receptor-binding agents, chemotherapeutics, growth factors, immunoglobulins, pharmaceuticals, nutraceuticals, antithrombogenic agents, antitumoral agents, antiangiogenic agents, anesthetics, vasodilation substances, wound healing agents, diagnostic agents, any other biologically-active substance known in the art, and combinations thereof.

Release of biologically-active agent A from base layer 18 occurs through erosion of base layer 18 when in contact with an aqueous fluid. Base layer 18 includes a release material to encourage erosion of base layer 18 and release of biologically-active agent A. As used herein, the term “erosion” (and variations thereof) means a disintegration (or decay) of base layer 18 due to a weakening of a structure of base layer 18 and/or a dissolving or disassociation of release material from base layer 18. Examples of release materials for incorporation in base layer 18 include inorganic crystalline materials, inorganic amorphous materials, organic crystalline materials, and organic amorphous materials. Examples of inorganic crystalline materials include calcium phosphate and calcium sulfate. The term “calcium phosphate” is used generically herein and includes inorganic substances such as, for example, dicalcium phosphate, tricalcium phosphate, tetracalcium phosphate, octacalcium phosphate, hydroxyapatite, and carbonate apatite.

In some embodiments, biologically-active agent A is released from base layer 18 pursuant to a substantially zero-order release kinetics, in which release of biologically-active agent A is generally independent of a concentration of biologically-active agent A in base layer 18. In some embodiments, the release profile of biologically-active agent A depends upon a surface area of base layer 18 or an erosion rate of base layer 18. In most embodiments, base layer 18 is configured to exhibit a relatively constant (or stable) rate of release of biologically-active agent A over a prolonged period of time (e.g., days, weeks, or months).

Polymer layer 20 includes a polymer material and biologically-active agent B. Polymer layer 20 can provide mechanical support for base layer 18 to, for example, discourage chipping or flaking of base layer 18. In some embodiments, polymer layer 20 is an erodible layer that erodes over time to expose base layer 18. In some embodiments, the polymer material forms a polymer matrix. The polymer matrix can serve as a reservoir for biologically-active agent B, which can be released from the polymer matrix through release mechanisms such as diffusion.

Polymer layer 20 can be optimized to control the release profile of biologically-active agent B. In some embodiments, biologically-active agent B is released from polymer layer 20 pursuant to substantially first-order release kinetics or substantially second order release kinetics. In some embodiments, polymer layer 20 exhibits a burst release profile over a limited duration of time (e.g., hours or days). In some of these embodiments, the burst release profile occurs as a result of hydration of polymer layer 20 and a resulting increase in sizes of pores included in polymer layer 20.

Examples of polymer materials for incorporation in polymer layer 20 include hydrophilic polymers, hydrophobic polymers, and polysaccharides. Examples of polymer materials for incorporation in polymer layer 20 include polyethylene, poly(ethylene oxide), poly(ethylene glycol), polyamino acids, poly(ethyloxazoline), polyvinyl-pyrrolidone, polyurethane, poly(vinyl alcohol) (PVA), polypropylene glycol, polyoxyethylene, polyacrylic acid, polyacrylamide, carboxymethyl cellulose, cellulose, dextrans, polysaccharides, starches, collagen, gelatins, biological polymers, chitin, vinyl acetate, polypropylene, polyacrylates, polycarbonate, polyamides, polyvinylchloride (PVC), polyetheretherketone (PEEK), polytetrafluroethylene (PTFE), polyoxymethylene, aromatic polymers, methacrylate polymers, polyethylene imine, glycerol, hyaluronan, and any combination or copolymer of these in any proportion.

Suitable materials for substrate 12 of article 10 include metals; metal alloys; protein films; synthetic or naturally-occurring organic or inorganic polymers such as polyethylene, polypropylene, polyacrylates, polycarbonate, polyamides, polyurethane, polyvinylchloride (PVC), polyetherketone (PEEK), polytetrafluroethylene (PTFE), PVA, polyoxymethylene, aromatic polymers, methacrylate polymers, cellulose, silicone and natural or synthetic rubbers; plastics; glass; ceramics; any other medical substrate material known in the art; derivatives of any of these; and any combination or copolymer of these in any proportion.

Article 10 may be a medical device (or healthcare product), a portion of a medical device, a material used to construct a medical device, or other healthcare products. Examples of medical devices that can be coated with compositions of the present invention include catheters such as urological catheters and central venous catheters; wound drains; orthopedic implants; external fixator pins used in orthopedic trauma; dental implants; feeding tubes; tracheal tubes; sutures; stents such as coronary stents and ureteral stents; percutaneous tubes; percutaneous nephrostomy tubes; medication delivery products such as needle-less connectors and/or IV products; and any other medical device or healthcare product that may contact bodily fluids.

Coating 14 can be tailored for various specific applications. This tailoring can be accomplished, for example, through selection of the biologically-active agent(s) included in coating 14 and optimization of the release profile of those biologically-active agent(s) from coating 14. The following discussion includes several illustrations of particular embodiments of coating 14 tailored for specific applications.

Coating 14 can be applied to medical devices that cause an acute pain profile followed by a chronic pain profile. In some embodiments, coating 14 may be configured to deliver an initial burst of analgesic/anesthetic from polymer layer 20 to address acute pain following implantation of article 10 and to deliver a stable and prolonged dosage of an analgesic/anesthetic from base layer 18 to address chronic pain. Medical devices that can result in an acute pain profile followed by a chronic pain profile include, for example, surgical wound drains, sutures, central venous catheters, percutaneous tubes, percutaneous nephrostomy tubes, ureteral stents, and other similar medical devices.

In some applications, a medical device (e.g., such as external fixator pins, wound drains, and endotracheal tubes) may need to be inserted into a microbially-contaminated space that can be expected to become less infected as systemic antibiosis is accomplished. In one embodiment, a medical device for use in such an application is coated with a coating 14 having an antimicrobial agent incorporated into base layer 18 for quick release and into polymer layer 20 for stable and prolonged release. As such, a burst of antimicrobial agent is released in the early stages of implantation to ward off early infection of the implantation site and, thereafter, the antimicrobial agent is released at a lesser, stable rate over a prolonged period of time.

In some applications, a medical device may need to be inserted into a location of a host subject in a manner that will cause pain to the host subject and also require protection from the microbial flora of the host subject. In one embodiment, a medical device for use in such an application is coated with a coating 14 including an analgesic/anesthetic in polymer layer 20 for quick release and an antibiotic in base layer 18 for stable and prolonged release.

Compositions of the present invention can be applied to surface 16 of substrate 12 in various manners to form coating 14. In most embodiments, surface 16 is first cleaned using, for example, water rinses, organic solvents, sonication, ultrasonic cleaners, detergents, any other cleaning means known in the art, or combinations of these. Base layer 18 is applied to surface 16 through deposition of release material and biologically-active agent from a liquid solution or suspension. Base layer 18 can be formed through one deposition step or a plurality of deposition steps. In some embodiments, one or more seed layers of release material are first deposited on surface 16 before depositing a subsequent layer(s) that includes both release material and biologically-active agent.

After application of base layer 18, polymer layer 20 is then applied to base layer 18 using any of the standard coating methods known in the art such as, for example, dipping, spraying, or rolling substrate 12 in a coating composition including the polymer material. Polymer layer 20 can be applied using one deposition step or a plurality of deposition steps. Polymer layer 20 may be cross-linked with a cross-linking agent such as, for example, an aldehyde or dialdehyde cross-linking agent. The extent of cross-linking can be used to control the release profile of biologically-active agents from polymer layer 20.

Cross-linking of polymer layer 20 can be achieved through any method known in the art. In some embodiments, polymer layer 20 is sprayed with a cross-linking agent. In some embodiments, the cross-linking agent is applied either between coating steps (if polymer layer 20 is applied through multiple coating steps) and/or after application of polymer layer 20 has been completed.

Base layer 18 and/or polymer layer 20 can each be dried after application of the final (or only) constituent layer of base layer 18 or polymer layer 20, between applications of constituent layers of base layer 18 or polymer layer 20, or combinations of these. Examples of suitable drying processes include air drying, infrared-radiation drying, convection or radiation drying (e.g., using a drying oven), forced-air drying (e.g., using a heat gun), or any combination of these.

In most embodiments, surface 16 is functionalized before application of base layer 18. Functionalization is a process by which chemical functional groups are added to surface 16 and/or existing functional groups on surface 16 of substrate 12 are modified. The functional groups can assist in the deposition of base layer 18 on surface 16 and affect the physical and mechanical properties of base layer 18. In some embodiments, the functional groups serve as nucleation sites for growth of base layer 18 on surface 16. For further discussion regarding functionalization procedures, see U.S. Pat. Nos. 5,759,708 and 5,958,430. Examples of functional groups that may be incorporated on surface 16 include carboxylates, sulfonates, phosphates, alkyls, alkenes, alkynes, aryl, alkylaryl, amines, hydroxyl, thiol, silyl, phosphoryl, cyano, metallocenyl, carboxyl, polyphosphates, and aldehyde groups.

Functionalization can be achieved using physical means, chemical means, or a combination of physical and chemical means. For example, in some embodiments, a plasma chamber is used to functionalize surface 16, whereby radio frequency excitation energy and a process gas (such as, for example, (3-glycidyloxypropyl)trimethoxysilane) are combined in the presence of surface 16.

### EXAMPLES

The present invention is more particularly described in the following examples that are intended as illustrations only, since numerous modifications and variations within the scope of the present invention will be apparent to those skilled in the art. Unless otherwise noted, all parts, percentages, and ratios reported in the following examples are on a weight basis, all reagents used in the examples were obtained, or are available, from commercial chemical suppliers or may be synthesized by conventional techniques.

Examples 1-5 illustrate one embodiment of a method of the present invention for producing a stent having a multilayer, bupivacaine-eluting coating including a calcium phosphate base layer and a PVA polymer layer.

### Example 1

### Surface Functionalization

Polyurethane stents were sonicated in deionized water for 10 minutes. The stents were placed on racks in a plasma chamber and exposed to an oxygen plasma for 3 minutes to remove organic residues. The stents were then exposed to a silane-containing plasma for 3 minutes to functionalize the surfaces of the stents, whereby (3-glycidyloxypropyl)trimethoxysilane was volatilized in a flash evaporator and introduced into the plasma chamber through a flow of argon gas at about 100 mTorr. The stents were then soaked overnight in a 0.1 M sodium sulfite solution to further functionalize the surfaces of the stents. The next day the stents were removed from the sodium sulfite solution, rinsed with deionized water for 5 minutes, and dried in a hot air chamber. After drying, the stents were immersed in 0.1 M HCl for 5 minutes, rinsed with deionized water for 5 minutes and dried in the hot air chamber. The sodium sulfite and hydrochloric acid treatments convert the terminal epoxide group of the silane functional groups to a sulfonic acid, which promotes deposition of base layer materials.

### Example 2

### Initial Calcium Phosphate Deposition

A calcium phosphate solution including 1.5 mM KH₂PO₄, 1.5 mM Na₂HPO₄, and 5 mM CaCl₂ was prepared. The functionalized stents of Example 1 were immersed in the calcium phosphate solution for about one hour at a temperature of about 30° C. The stents were then rinsed in deionized water rinse for 5 minutes and dried in a hot air chamber. The purpose of this step was to provide an initial deposition of calcium phosphate.

### Example 3

### Deposition of Calcium Phosphate and Bupivacaine

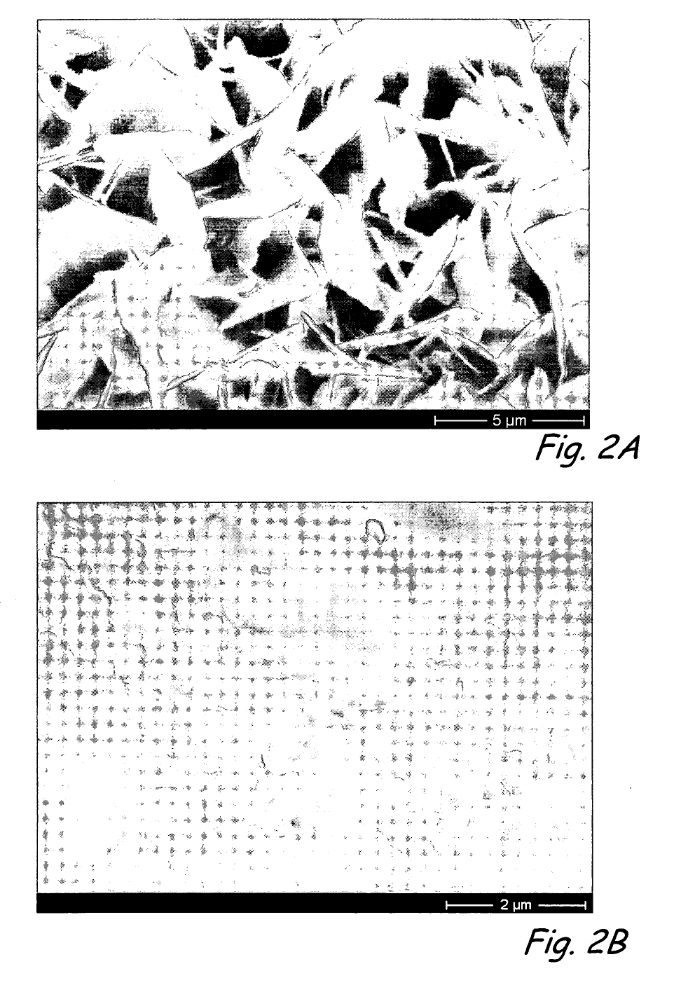

The stents of Example 2 were immersed for about one hour, at a temperature of about 30° C., in a calcium phosphate solution of Example 2 including 4 mM bupivacaine. The stents were then rinsed with deionized water for about 5 minutes and dried in a hot air chamber. This coating procedure was repeated five additional times. FIG. 2A shows a scanning electron micrograph of one of the resulting stents coated with a base layer of calcium phosphate and bupivacaine. As can be seen from FIG. 2A, the base layer is comprised of crystalline leaflets that have nucleated and grown from the substrate surface.

### Example 4

### Deposition of PVA

A PVA coating solution was prepared having a concentration of 5% PVA and 2% bupivacaine. The coated stents of Example 3 were sprayed with the PVA coating solution and dried for 24 hours. FIG. 2B shows a scanning electron micrograph of one of the resulting PVA-coated stents. As compared to the underlying calcium phosphate base layer shown in FIG. 2A, the overlying PVA layer of FIG. 2B is more uniform and continuous. As shown in FIG. 2B, the PVA layer imparts mechanical properties to the underlying calcium phosphate base layer.

### Example 5

### Bupivacaine Elution Profile

An elution analysis was performed to assay release of bupivacaine over time from the coated stents of Example 4. An 18 mm long test specimen having a known surface area was cut from one of the coated stents of Example 4 and placed in a flow chamber. For 7 days, the test specimen was bathed with 10 ml per day of a phosphate buffered saline (PBS) solution. Every 24 hours, a 100 μl sample was taken of the 10 ml of PBS effluent collected over the preceding 24 hour period. The amount of bupivacaine in a 10 μl aliquot of the sample was quantified using a reverse-phase high pressure liquid chromatography (HPLC) system including a 250 micron silica column. A solution of 30% acetonitrile and 70% buffered acid (985 ml of HPLC-grade water, 15 ml 1.0 M HCl, and 1.38 grams monobasic NaH₂PO₄) was employed as the mobile phase for the HPLC analysis. The data from the HPLC analysis was then normalized against the surface area of the test specimen and 10 ml volume of PBS effluent.

The results of the above tests are shown in FIG. 3. Each data point represents the amount of bupivacaine eluted into the PBS solution during the proceeding 24-hour period, with each data point expressed in terms of in μg of bupivacaine released per cm² of coated stent surface. For example, the data point for day 1 indicates the amount of bupivacaine released into solution during the first day (i.e., hours 0-24), while the data point for day 2 indicates the amount of bupivacaine released into solution during the second day (i.e., hours 24-48). As seen in FIG. 3, the relative burst release of bupivacaine occurring during days 1-3 is attributable to the release of bupivacaine from the polymer layer, while the more gradual release of bupivacaine occurring in days 4-7 is attributable to erosion of the calcium phosphate base layer. As such, the results of FIG. 3 illustrate the differing release kinetics of the base layer and the polymer layer exhibited by some embodiments of the present invention.

Thus, as described above, the composition and coatings of the present invention provide a vehicle for delivering one or more biologically-active agents using release kinetics that can be tailored to various applications.

Although the present invention has been described with reference to preferred embodiments, workers skilled in the art will recognize that changes may be made in form and detail without departing from the spirit and scope of the invention.

1. An article capable of affecting a biological process, the article comprising:
a substrate having a surface;
an erodible layer positioned over the surface of the substrate and comprising a release material and a first biologically-active agent releasable from the erodible layer when exposed to an aqueous fluid; and
a polymer layer comprising a polymer and a second biologically-active agent releasable from the polymer layer when exposed to the aqueous fluid.

2. The article of claim 1, wherein the first and second biologically-active agents are the same biologically-active agent.

3. The article of claim 1, wherein the release material is selected from the group consisting of inorganic crystalline materials, inorganic amorphous materials, organic crystalline materials, and organic amorphous materials.

4. The article of claim 1, wherein the release material is selected from the group consisting of calcium phosphate and calcium sulfate.

5. The article of claim 1, wherein the polymer is selected from the group consisting of hydrophilic polymers, hydrophobic polymers, and polysaccharides.

6. The article of claim 1, wherein the first and second biologically-active agents are selected from the group consisting of analgesics, antimicrobials, anti-inflammatories, targeting agents, cytokines, immunotoxins, antihistamines, receptor-binding agents, chemotherapeutics, growth factors, immunoglobulins, pharmaceuticals, nutraceuticals, antithrombogenic agents, antitumor agents, antiangiogenic agents, anesthetics, vasodilation substances, wound healing agents, diagnostic agents, and any combination of these.

7. The article of claim 1, wherein the article is configured to release the second biologically-active agent, upon exposure to the aqueous fluid, more rapidly than the first biologically-active agent.

8. The article of claim 1, wherein release kinetics of the first biologically-active agent depends on a rate of erosion of the erodible layer when exposed to the aqueous fluid.

9. The article of claim 1, wherein release kinetics of the second biologically-active agent depends on a rate of diffusion of the second biologically-active agent.

10. The article of claim 1, wherein the surface of the substrate includes functional groups that function as attachment sites for the erodible layer.

11. The article of claim 1, wherein the polymer layer mechanically reinforces the erodible layer.

12. The article of claim 1, wherein the erodible layer is in contact with polymer layer.

13. The article of claim 1, wherein the erodible layer is in contact with the substrate surface.

14. A composition for affecting a biological process, the composition comprising:
a release material that disintegrates in an aqueous fluid;
a first biologically-active agent incorporated within the release material and releasable through disintegration of the release material;
a polymer matrix; and
a second-biologically active agent incorporated within the polymer matrix and releasable from the polymer matrix upon exposure to an aqueous fluid.

15. The composition of claim 14, wherein the first biologically-active agent is releasable from the release material with substantially zero-order release kinetics.

16. The composition of claim 14, wherein the second biologically-active agent is releasable from the polymer matrix with substantially first-order release kinetics.

17. The composition of claim 14, wherein the second biologically-active agent is releasable from the polymer matrix with substantially second-order release kinetics.

18. The composition of claim 14, wherein the composition is configured to release, upon exposure to the aqueous fluid, the second biologically-active agent more rapidly than the first biologically-active agent.

19. The composition of claim 14, wherein the first and second biologically-active agents are the same biologically-active agent.

20. The composition of claim 14, wherein the release material is selected from the group consisting of inorganic crystalline materials, inorganic amorphous materials, organic crystalline materials, and organic amorphous materials.

21. The composition of claim 14, wherein the release material is selected from the group consisting of calcium phosphate and calcium sulfate.

22. The composition of claim 14, wherein the first and second biologically-active agents are selected from the group consisting of analgesics, antimicrobials, anti-inflammatories, targeting agents, cytokines, immunotoxins, antihistamines, receptor-binding agents, chemotherapeutics, growth factors, immunoglobulins, pharmaceuticals, nutraceuticals, antithrombogenic agents, antitumor agents, antiangiogenic agents, anesthetics, vasodilation substances, wound healing agents, diagnostic agents, and any combination of these.

23. The composition of claim 14, wherein the polymer matrix is in contact with the release material.

24. A method for coating a substrate, the method comprising:
providing functional groups on a surface of the substrate;
depositing a base layer on the functionalized surface, the base layer comprising:
a release material; and
a first biologically-active agent; and
forming a polymer layer over the base layer, the polymer layer including a second biologically-active agent that is releasable from the polymer layer when exposed to an aqueous fluid.

25. The method of claim 24, wherein the first and second biologically active-agents are the same biologically-active agent.

26. The method of claim 24, wherein, after the polymer layer is formed over the base layer, the polymer layer is immersed in a solution including the second-biologically active agent.

27. The method of claim 24, and further comprising:
mixing the second biologically-active agent and the polymer before forming the polymer layer over the base layer.

28. The method of claim 24, wherein the first and second biologically-active agents are selected from the group consisting of analgesics, antimicrobials, anti-inflammatories, targeting agents, cytokines, immunotoxins, antihistamines, receptor-binding agents, chemotherapeutics, growth factors, immunoglobulins, pharmaceuticals, nutraceuticals, antithrombogenic agents, antitumor agents, antiangiogenic agents, anesthetics, vasodilation substances, wound healing agents, diagnostic agents, and any combination of these.

29. The method of claim 24, wherein depositing a base layer comprises immersing the functionalized surface of the substrate in a coating solution including the release material and the first-biologically-active agent.

30. The method of claim 24, wherein the polymer layer is in contact with the base layer.
